(19) United States
(12) Patent Application Publication
(10) Pub. No.: US 2025/0213201 A1
(43) Pub. Date: Jul. 3, 2025

(54) PATIENT SUPPORT APPARATUS HAVING CONTACT FREE PATIENT TEMPERATURE MEASUREMENT

(71) Applicant: Hill-Rom Services, Inc., Batesville, IN (US)

(72) Inventors: Nicholas Comparone, Batesville, IN (US); Vijay Aditya Tadipatri, Batesville, IN (US)

(21) Appl. No.: 18/987,943

(22) Filed: Dec. 19, 2024

Related U.S. Application Data
(60) Provisional application No. 63/616,866, filed on Jan. 2, 2024.

(51) Int. Cl. A61B 5/00; A61B 5/0205

(52) U.S. Cl. CPC A61B 5/746; A61B 5/02055; A61B 5/6892; A61B 5/7246; A61B 5/7465

(57) ABSTRACT

A patient support apparatus includes a diagnostic system that collects patient vital signs in real-time to determine the patient's early warning score and a notification system that alerts caregivers if the early warning score exceeds a threshold. The patient support apparatus may change operation in an effort to mitigate an early warning score that exceeds a threshold.

### CROSS-REFERENCE TO RELATED APPLICATION

This application claims the benefit of U.S. Patent Application No. 63/616,866, filed Jan. 2, 2024, the entire disclosure of which is incorporated hereby reference in its entirety.

### BACKGROUND

The present disclosure is directed to a patient support apparatus having an integrated diagnostic system that determines and responds to patient vital signs.

It has been determined that the interrelationship between patient vital signs can be monitored to provide a unified identification of the degradation of the patient's health. Current systems rely on the vitals to be measured on the patients and then using a modified early warning score (MEWS) or a system inflammatory response syndrome (SIRS) score to identify the patient's acuity. Since these methods rely on a manual recording of patient's vitals the cadence of early warning scores is constrained by nurse's rounding. This limits the timing of the determination of such a score and increases the time of response to a patient who is degrading.

Monitoring key vital signs in real-time is difficult due to the typical need to contact the patient to get heart rate, respiratory rate, and temperature readings. While sensors that maintain contact with a patient are known, such sensors limit mobility of the patient due to the tethered nature of the related sensors and sensing devices. However, as the progression of care pushes for more mobility for patients during recovery, there is a need to provide real-time monitoring without contacting the patient. Even so, the apparently healthy and mobile patients can degrade quickly when they become septic. Thus, there is a need for a real-time monitoring system that provides vital signs in real-time and monitors those vitals for evidence of patient degradation indicative of the onset of sepsis or other conditions.

### SUMMARY

The present disclosure includes one or more of the features recited in the appended claims and/or the following features which, alone or in any combination, may comprise patentable subject matter.

According to a first aspect of the present disclosure, a patient support apparatus comprises a diagnostic system including a cardiopulmonary monitor and a temperature monitor. The cardiopulmonary monitor detects a patient's heart rate and respiratory rate without contacting the patient. The temperature monitor includes a targeting sensor and an infrared sensor, and a controller. The controller processes data from the targeting sensor to determine a location of a feature of a patient supported on the patient support apparatus and processes data from the infrared sensor in the location of the feature of the patient to determine the patient's body temperature. The controller utilizes data from the cardiopulmonary monitor and the patient's body temperature to establish an early warning score for the patient. The diagnostic system compares the early warning score for the patient to a threshold early warning score indicative of an imminent adverse event, and, if the early warning score exceeds the threshold, provides an output initiate a therapeutic intervention to address the patient's condition.

In some embodiments of the first aspect, the therapeutic intervention to address the patient's condition includes altering a function of the patient support apparatus.

In some embodiments of the first aspect, the targeting sensor comprises a time of flight sensor.

In some embodiments of the first aspect, the feature of the patient includes the patient's ear.

In some embodiments of the first aspect, the feature of the patient includes the patient's forehead.

In some embodiments of the first aspect, the temperature monitor is mounted to a movable siderail of the patient support apparatus.

In some embodiments of the first aspect, the feature of the patient is detected using a machine learning algorithm.

In some embodiments of the first aspect, the early warning score is displayed on a graphical user interface of the patient support apparatus.

In some embodiments of the first aspect, the early warning score is transmitted to a central monitoring station.

In some embodiments of the first aspect, the early warning score is displayed on a central monitoring station that is positioned remotely from the patient's location.

In some embodiments of the first aspect, the output signaling to a caregiver to initiate a therapeutic intervention to address the patient's condition is displayed on a graphical user interface of the patient support apparatus.

In some embodiments of the first aspect, the output signaling to a caregiver to initiate a therapeutic intervention to address the patient's condition is transmitted to a central monitoring station.

In some embodiments of the first aspect, the output signaling to a caregiver to initiate a therapeutic intervention to address the patient's condition is displayed on a dashboard that is positioned remotely from the patient's location.

In some embodiments of the first aspect, the diagnostic system determines if the temperature monitor is correctly positioned before attempting to determine a location of a feature of the patient.

In some embodiments of the first aspect, the temperature system is positioned on a movable portion of the patient support apparatus and the diagnostic system identifies the position of the movable portion of the patient support apparatus to determine if the temperature monitor is correctly positioned before attempting to determine a location of a feature of the patient.

In some embodiments of the first aspect, the cardiopulmonary monitor is positioned on a frame member of the patient support apparatus.

In some embodiments of the first aspect, the cardiopulmonary monitor is positioned beneath a mattress supporting a patient on the patient support apparatus.

In some embodiments of the first aspect, the heart rate detected by the cardiopulmonary monitor is displayed on a graphical user interface of the patient support apparatus.

In some embodiments of the first aspect, the respiration rated detected by the cardiopulmonary monitor is displayed on a graphical user interface of the patient support apparatus.

In some embodiments of the first aspect, the diagnostic system comprises a processor, the processor operable to receive signals from the cardiopulmonary monitor indicative of the heart rate and the respiratory rate of the patient, signals from the infrared sensor indicative of the patient's temperature, and process the signals according to a predetermined relationship to determine the patient's early warning score in real time.

According to a second aspect of the present disclosure, a method of monitoring a patient supported on a patient support apparatus comprises: receiving from a contactless cardiopulmonary monitor signals indicative of the heart rate and the respiratory rate of a patient supported on a patient support apparatus; operating a temperature monitor positioned on a movable portion of the patient support apparatus by detecting a field of view using a detector detecting in the visible spectrum, using a time-of-flight calculation to determine the distance of a patient in the field-of-view from the detector, identifying a patient feature from the field of view, and targeting the detected feature with an infrared sensor to determine a core temperature of the patient; utilizing the heart rate, respiratory rate, and temperature of the patient to determine an early warning score for the patient in real-time; and if the early warning score exceeds a predetermine threshold, causing an intervention to mitigate the patient's condition.

In some embodiments of the second aspect, the step of identifying the patient feature from the field of view comprises applying a machine learning algorithm.

In some embodiments of the second aspect, the step of causing an intervention to mitigate the patient's condition includes altering a function of the patient support apparatus.

In some embodiments of the second aspect, the step of causing an intervention to mitigate the patient's condition includes prompting a caregiver to intervene to provide a therapy to the patient.

In some embodiments of the second aspect, the step of causing an intervention to mitigate the patient's condition includes prompting a caregiver to intervene to provide a therapy to the patient.

In some embodiments of the second aspect, the feature of the patient includes the patient's ear.

In some embodiments of the second aspect, the feature of the patient includes the patient's forehead.

In some embodiments of the second aspect, the temperature monitor is mounted to a movable siderail of the patient support apparatus.

In some embodiments of the second aspect, the feature of the patient is detected using a machine learning algorithm.

In some embodiments of the second aspect, the early warning score is displayed on a graphical user interface of the patient support apparatus.

According to a third aspect of the present disclosure, an early warning score monitoring system for a patient supported on a patient support apparatus comprises a diagnostic system and a notification system. The diagnostic system includes a contactless cardiopulmonary monitor providing signals indicative of the heart rate and the respiratory rate of the patient. The diagnostic system also includes a temperature monitor positioned on a movable portion of the patient support apparatus. The temperature monitor providing a signal indicative of the core temperature of the patient. The diagnostic system determines an early warning score of the patient in real-time. The notification system provides a notification to a caregiver if the real-time early warning score exceeds a predetermined threshold.

In some embodiments of the third aspect, the temperature monitor includes a targeting sensor and an infrared sensor.

In some embodiments of the third aspect, the targeting sensor comprises a time of flight sensor.

In some embodiments of the third aspect, the targeting sensor identifies a feature of the patient and the temperature monitor identifies a temperature of the patient at the feature using the infrared sensor. In some embodiments of the third aspect, the feature of the patient includes the patient's ear. In some embodiments of the third aspect, the feature of the patient includes the patient's forehead.

In some embodiments of the third aspect, the temperature system is positioned on a movable portion of the patient support apparatus and the diagnostic system identifies the position of the movable portion of the patient support apparatus to determine if the temperature monitor is correctly positioned before attempting to determine a location of a feature of the patient.

In some embodiments of the third aspect, the diagnostic system determines if the temperature monitor is correctly positioned before attempting to determine a location of a feature of the patient.

In some embodiments of the third aspect, the cardiopulmonary monitor is positioned on a frame member of the patient support apparatus.

In some embodiments of the third aspect, the cardiopulmonary monitor is positioned beneath a mattress supporting a patient on the patient support apparatus.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view of a patient support apparatus of the present disclosure;

FIG. 2 is a perspective of a patient support apparatus similar to FIG. 1, the patient support apparatus FIG. 2 shown without a mattress to illustrate the mounting of a cardiopulmonary monitor on a frame member beneath the mattress;

FIG. 3 is a block diagram of a system according to the present disclosure;

FIG. 4 is a block diagram of a temperature monitor according to the present disclosure;

FIG. 5 is an illustration of a portion of a siderail of the FIG. 1 showing the mounting of the temperature monitor on the siderail;

FIGS. 6-8 are illustrations of a field-of-view generated by the temperature monitor of FIGS. 4 and 5 showing the manner in which the field-of-view is divided during the application of a feature identification algorithm;

FIG. 9 is a flowchart of an algorithm used by the temperature monitor of FIGS. 4-5 to determine the temperature of a patient supported on the patient support apparatus of FIG. 1;

FIG. 10 is a flowchart of an algorithm used by the cardiopulmonary monitor to detect and log a patient's heart rate and respiratory rate;

FIG. 11 is a flowchart of an algorithm used by a diagnostic system of the present disclosure to determine and respond to an early warning score for a patient; and

FIG. 12 is a view of a siderail of the patient support apparatus of FIG. 1 with portions enlarged to show the output of the diagnostic system on a user interface.

### DETAILED DESCRIPTION

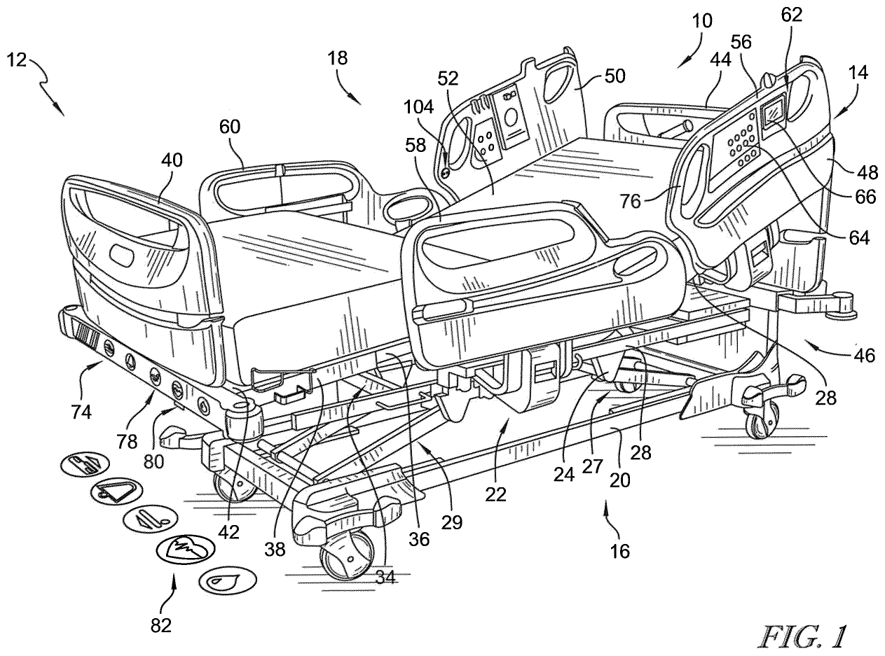

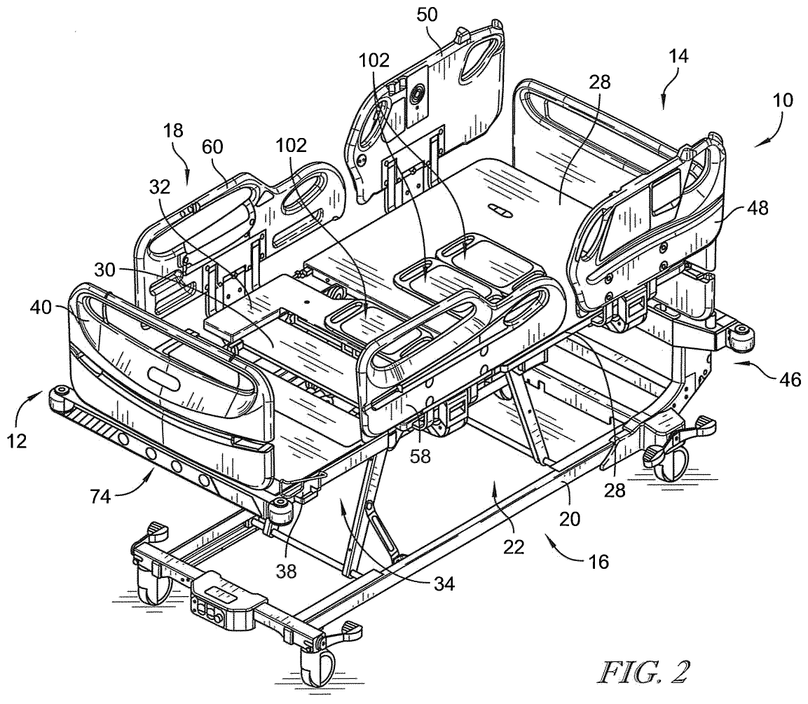

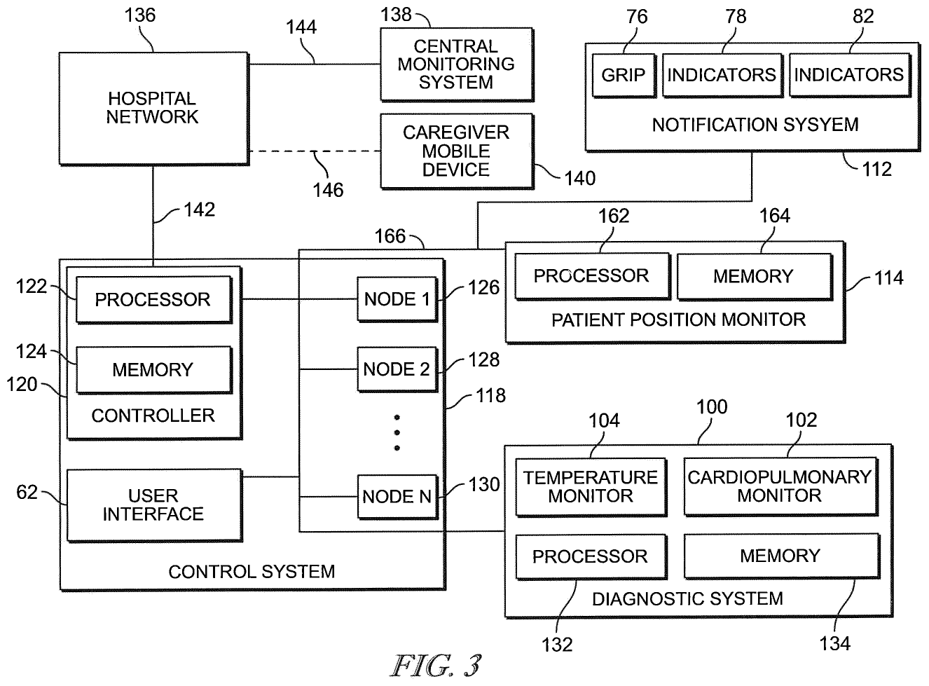

Referring to FIG. 1, a patient support apparatus 10 is illustratively embodied as a hospital bed 10. The hospital bed 10 includes an integrated diagnostic system 100 (shown in the block diagram of FIG. 3), the diagnostic system 100 including a cardiopulmonary sensor 102 mounted to a deck section of the hospital bed 10 as suggested in FIG. 2 and a temperature sensor 104 positioned on a siderail 50 as shown in FIG. 1. The diagnostic system 100 operates as a real-time vital signs monitoring system, which, as discussed below, is operable to monitor a patient supported on the hospital bed 10 to provide real-time analysis of a patient's early warning score (EWS).

The view shown in FIG. 1 is generally taken from a position that is oriented at the left side, foot end of the hospital bed 10. For purposes of orientation, the discussion of the hospital bed 10 will be based on the orientation of a patient supported on the hospital bed 10 in a supine position. Thus, the foot end 12 of the hospital bed 10 refers to the end nearest the patient's feet when the patient is supported on the hospital bed 10 in the supine position. The hospital bed 10 has a head end 14 opposite the foot end 12. A left side 16 refers to the patient's left when the patient is lying in the hospital bed 10 in a supine position. The right side 18 refers to the patient's right. When reference is made to the longitudinal length of the hospital bed 10, it refers a direction that is represented by the lines that generally extend between the head end 14 and foot end 12 of the hospital bed 10. Similarly, lateral width of the hospital bed 10 refers to a direction that is represented by the lines that generally extend between the left side 16 and right side 18.

The hospital bed 10 includes a base frame 20 which supports a lift system 22. The lift system 22 engages the base and an upper frame 24 such that the lift system 22 moves the upper frame 24 vertically relative to the base frame 20. The lift system 22 includes a head end linkage 27 and a foot end linkage 29. Each of the linkages 27 and 29 are independently operable and may be operated to cause the hospital bed 10 to move into a tilt position which is when the head end 14 of the upper frame 24 is positioned lower than the foot end 12 of the upper frame 24. The hospital bed 10 may also be moved to a reverse tilt position with the foot end 12 of the upper frame 24 is positioned lower than the head end 14 of the upper frame 24.

The upper frame 24 supports a load frame 26. The load frame 26 supports a head deck 28 which is movable relative to the load frame 26. The load frame 26 also supports an articulated seat deck 30 (seen in FIG. 3), also movable relative to the load frame 26 and a fixed seat deck 32 (also seen in FIG. 3). Also supported from the load frame 26 is a foot deck 34 that is articulated and moveable relative to the load frame 26. The foot deck 34 in the illustrative embodiment of FIG. 1 provides for powered pivoting of the foot deck 34 and manual extension and retraction of the foot deck 34 to vary the length of the foot deck 34. In other embodiments, powered pivoting of the foot deck 34 may be omitted and the related movement may be caused manually, or follow movement of the articulated seat deck 30. In addition, in some embodiments, extension and retraction of the foot deck 34 may be powered by an actuator.

The foot deck 34 includes a first portion 36 and a second portion 38, which moves relative to the first portion 36 to vary the size of the foot deck 34. The second portion 38 moves generally longitudinally relative to the first portion 36 to vary the longitudinal length of the foot deck 34 and, thereby, the longitudinal length of the hospital bed 10.

A foot panel 40 is supported from the second portion 38 and extends vertically from an upper surface 42 of the second portion 38 to form a barrier at the foot end 12 of the hospital bed 10. A head panel 44 is positioned on an upright structure 46 of the base frame 20 and extends vertically to form a barrier at the head end 14 of the hospital bed 10. A left head siderail 48 is supported from the head deck 28 and is moveable between a raised position shown in FIG. 1 and a lowered position as is known in the art. A right head siderail 50 is also moveable between the raised position of FIG. 1 and lowered position. As shown in FIG. 1, in the raised position, the siderails 48 and 50 extend above an upper surface 52 of a mattress 54 of the hospital bed 10 when the siderails 48 and 50 are in a raised position. In a lowered position an upper edge 56 of the left head siderail 48 is below the upper surface 52.

The hospital bed 10 also includes a left foot siderail 58 and a right foot siderail 60, each of which is supported directly from the load frame 26. Each of the siderails 48, 50, 58, and 60 are operable to be lowered to a position below the upper surface 52. It should be noted that when the head deck 28 is moved, the head siderails 48 and 50 move with the head deck 28 so that they maintain their relative position to the patient. This is because both of the head siderails 48 and 50 are supported by the head deck 28.

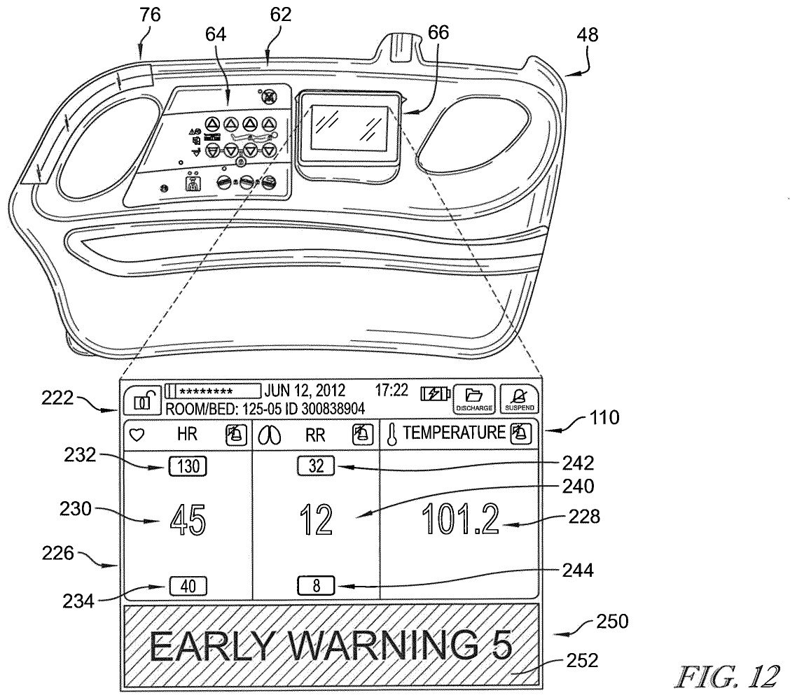

Referring to the left head siderail 48 shown in FIG. 12, a user interface 62 includes a hard panel 64 and a graphical user interface 66. The user interface 62 will be discussed in further detail below, but it should be understood that the hard panel 64 provides indications to a user regarding the status of certain functions of the hospital bed 10 as well as providing a standard set of fixed input devices. The graphical user interface 66 includes a touchscreen display 110 that provides information to a user as well as allowing for flexible, menu driven, operation of certain functions of the hospital bed 10. The graphical user interface 66, also known as a flip-up display (FUD), is mounted to the siderail 48 with a pivotable connection so that the graphical user interface 66 may be pivoted to allow a user the more easily view and interact with the graphical user interface 66, as is known in the art. In some embodiments, the right head siderail 50 may include a second graphical user interface duplicative of the graphical user interface 66.

Additional information is provided to a caregiver through an optional indicator panel 74 which displays the status of various conditions of the hospital bed 10 graphically to a caregiver at the foot end 12 of the hospital bed 10. The location of the indicator panel 74 makes the statuses of the conditions easily discernable from a distance, such that a caregiver may quickly ascertain the statuses from the hallway or the door of a patient's room. As will be discussed below, additional indication of the statuses may be projected on the floor under the foot end 12 of the hospital bed 10, providing larger images on the floor, making the images more easily discerned by a caregiver. Similarly, an illuminated grip 76 is positioned on the left head siderail 48, the illuminated grip 76 being selectively illuminated in different colors to provide an indication of the status of one or more functions of the hospital bed 10 to a caregiver. In some embodiments, the right head siderail 50 also includes an illuminated grip similar to the illuminated grip 76 and which includes the functionality of the illuminated grip 76.

The head end side rails 48 and 50 are configurable to provide additional indications of the status of components of the hospital bed 10 under the control of a notification system 112 by illuminating the grip 76 of the head siderails 48, 50, indicators 78 displayed on the panel 74 on the foot end 12 of the bed 10, and indicators 82 illuminated on the floor beneath the foot end 12 of the bed 10. The present disclosure includes the notification system 112 (shown in FIG. 3) as described herein, but in some embodiments, the notification system 112 may further include the functionality to illuminate the grip 76 is similar to that disclosed in WO2016/196403, filed May 29, 2016, titled “PATIENT SUPPORT APPARATUS,” and incorporated by reference herein for the disclosure of a notification system and for a structure for illuminating a grip of a siderail.

In operation the grip 76 has four states, not illuminated, illuminated in a blue color, illuminated in an amber color, or illuminated in a red color. In the current embodiment, the grip 76 is not illuminated in one of two conditions: if a patient position monitoring system 114 is disarmed and a patient is in hospital bed 10, or if the patient position monitoring system is armed and the patient is in the proper position in the bed 10. The patient monitoring system 114 uses signals from a scale system to detect the presence and movement of a patient on the hospital bed 10 in the manner disclosed in WO2016/196403 incorporated above. The grip 76 is illuminated blue if the patient position monitoring system 114 is disarmed and the patient is out of the hospital bed 10. The blue illumination tends to provide additional lighting for the patient if the ambient light is relatively low. The grip 76 is illuminated in an amber color if the patient position monitoring system 114 is armed and the patient is not in the proper position on the hospital bed 10. This amber illumination provides an additional indication to a caregiver of the alarm condition of the patient position monitoring system 114. The grip 76 is illuminated in a red color if the diagnostic system 100 is in an alarm state, thus, providing an additional indication to a caregiver of the alarm condition of the diagnostic system 100.

Referring now to FIG. 3, the diagnostic system 100 is connected to the user interface 62 via a communications link 116 which, in the disclosed embodiment, is a wired communication link. In some embodiments, the link 116 could be embodied as a wireless communication link. The user interface 62 is shown to be part of a frame control system 118 which controls the functionality of the frame components of the hospital bed 10 as is known in the art. The frame control system 118 is shown to include a controller 120 that has a processor 122 and a memory device 124, the memory device 124 including instructions that, when executed by the processor 122 control the functionality of various nodes 126, 128, and 130 of the frame control system 118. The nodes 126, 128, and 130 are representative only and the frame control system 118 may include any of a number of nodes associated with various functions of the hospital bed 10, such as controlling the operation of one or more drives, a scale system, an air system providing functionality to the mattress 52, or other nodes dedicated to functions of the hospital bed 10 as is known in the art. Each of the nodes 126, 128, and 130 may include a separate processor and memory device that contains instructions that when executed by the processor cause the particular node 126, 128, or 130 to perform a specific function of the respective node 126, 128, or 130.

In the present embodiment, the controller 120 and the nodes 126, 128, and 130 are peers on a peer-to-peer network. In some other embodiments, the controller 120 may operate as a server and each of the nodes 126, 128, and 130 function as clients. According to the present disclosure, the control system 118 includes the notification system 112, the patient position monitor 114, the diagnostic system 100, the user interface 62, the controller 120, and the functional nodes 126, 128, and 130. Illustratively, the components of the control system are interconnected via a bus 166 which permits serial communications between the various components of the control system 118. The notification system 112 comprises the illuminated grip 76, the indicators 78, and the indicators 82. The notification system 112 is a functional module which may utilize the controller 120 or any of the other nodes of the control system 118 to define the operation of the grip 76, indicators 78, and indicators 82, with the controller 120 providing the logic and processing functionality to determine the particular state of the notification system 112. For example, the patient position monitor 114, may use information from a scale system or other patient sensing functionality of one of the nodes 126, 128, 130, process that information using instructions and a memory device 164 that, when executed by a processor 162, provides the logical operation of the patient position monitor 114.

The controller 120 of the control system 118 is connected to a hospital network 136 by link 142. The hospital network 136 may include one or more servers as is known in the art. According to the present disclosure, the hospital network 136 communicates with a central monitoring system 138 via a communication link 144. According to the present disclosure, the communication link 144 is a wired link. Additionally, the hospital network 136 is operable to communicate with one or more caregiver mobile devices 140 through a wireless communication link 146. The structure allows the hospital network 136 to share information regarding the status of the hospital bed 10 the central monitoring system 138 that is spaced apart from a room in which the particular hospital bed 10 is located in real-time. Similarly, the status of components of the control system 118 may also be shared with the caregiver mobile device 140 through the hospital network 136 so that a caregiver who is spaced apart from the particular location of the hospital bed 10 may receive updates regarding the status of the hospital in 10 real-time.

According to the present disclosure, diagnostic system 100 includes the temperature monitor 104, the cardiopulmonary monitor 102, a processor 132, and a memory 134, the memory 134 including instructions that, when executed by the processor 132, analyze the signals of the modern 104 and the cardiopulmonary module 102 to determine vital signs of the patient supported on the hospital bed 10. According to the present disclosure, the temperature monitor 104 determines a core temperature of the patient. The cardiopulmonary monitor 102, determines both the respiratory rate and the heart rate of the patient. As will be described below, this information is useful in providing real-time monitoring of the patient such that the patient's vital signs can be regularly charted in their medical record over the hospital network 136. Additionally, Inc. present disclosure, this real-time monitoring of these vital signs permits the vital signs to be evaluated according to a predetermined algorithm to establish a real-time EWS for the patient. If the EWS moves to a value which indicates that the patient is in distress or at risk of developing a dangerous condition, that information can be communicated via the notification system 112 so that a caregiver is prompted to intervene and provide the patient with either a prophylactic intervention to reverse the progression by the patient or to provide a therapy to treat the patient's condition identified by the EWS. In some applications, the EWS may be considered by the controller 120 such that the controller 120 operates one or more functions of the hospital bed 10 to provide a treatment to the patient. For example, in some embodiments, one of the nodes 126, 128, or 130 may control the function of a therapeutic device which provides a therapy such as continuous lateral rotation therapy or percussions/vibration therapy to the patient. The controller 120 may be operable to begin such a therapy based on the EWS identified by the diagnostic system 100. In some cases, the controller 120 may cause the head section 28 of the hospital bed 10 to raise to mitigate a heart rate or respiratory rate problem being experienced by the patient.

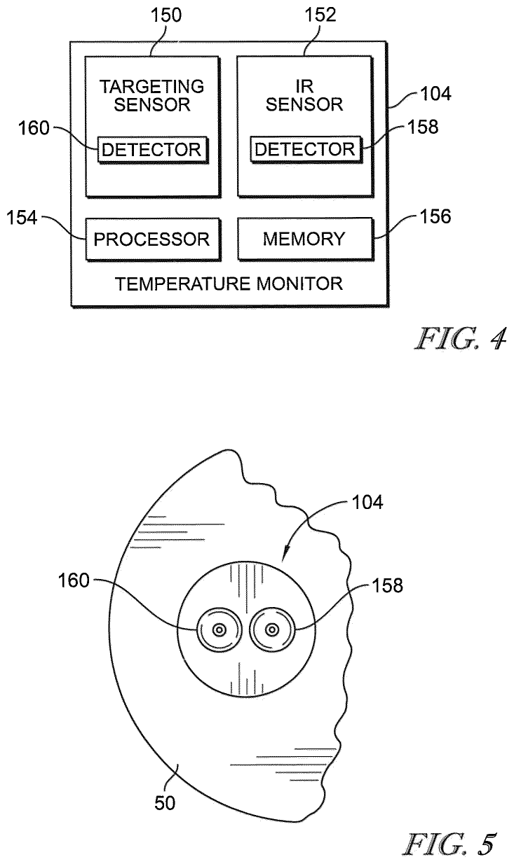

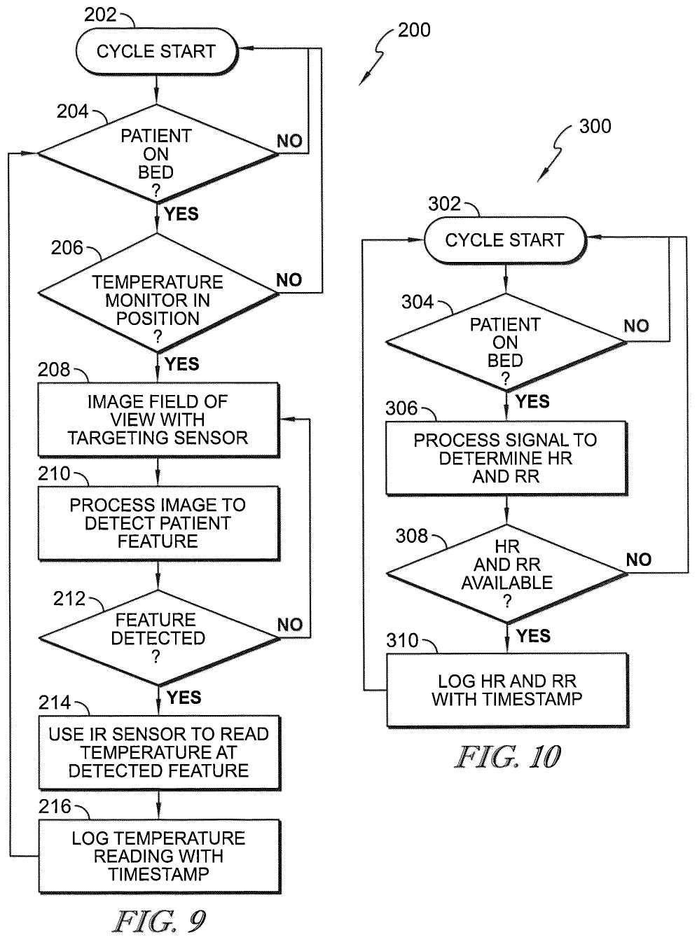

Referring now to FIGS. 4 and 5, the temperature monitor 104 includes a targeting sensor 150 and an infrared sensor 152. The temperature monitor 104 also includes a dedicated processor 154 and memory 156, the memory 156 including instructions that, when executed by the processor 154, control the operation of the targeting sensor 150 infrared sensor 152 according to the algorithm 200 shown in FIG. 9 acquire a signal indicative of the court temperature of the patient supported on the hospital bed 10.

The targeting sensor 150 includes a detector 160 that operates in the visible spectrum to acquire image data that is processed by the processor 154 to identify features of a patient based on a machine learning model that has been taught identify the features of the patient in a hospital bed 10. The detector 160 is shown mounted to the right hand side rail 50 of the hospital bed 10 in FIG. 5. Similarly, the infrared sensor 152 includes a detector 158 that is controlled by the processor 154 to detect a particular region of interest on the patient once the patient features have been identified from the information from the targeting sensor 150. The processor 154 processes the signal from the infrared sensor 152 to determine a value for the core temperature of the patient based on empirical modeling.

The process 200 of FIG. 9 it is a continuous loop that begins with a cycle start at step 202. The process advances to a decision step 204 where information from the patient positioned monitor 114 is identified to determine whether or not a patient is present on the hospital bed 10. If the patient is determined not to be on the hospital bed 10, the process 200 returns to step 202 and repeats until patient is detected. If the patient is detected on the hospital bed 10 at step 204, the process advances to a decision step 206 which determines whether or not the temperature monitor 104 is in a position that allows a temperature to be taken from the patient. For example, if the system determines that the side rail 50 is a lowered position such that the temperature monitor 104 is below the level of the mattress 52, then a temperature cannot be taken. If that is the case, then the process 200 returns to step 202 and continues to cycle. However, if the temperature monitor is determined to be in position at step 206, the process advances to process step 208.

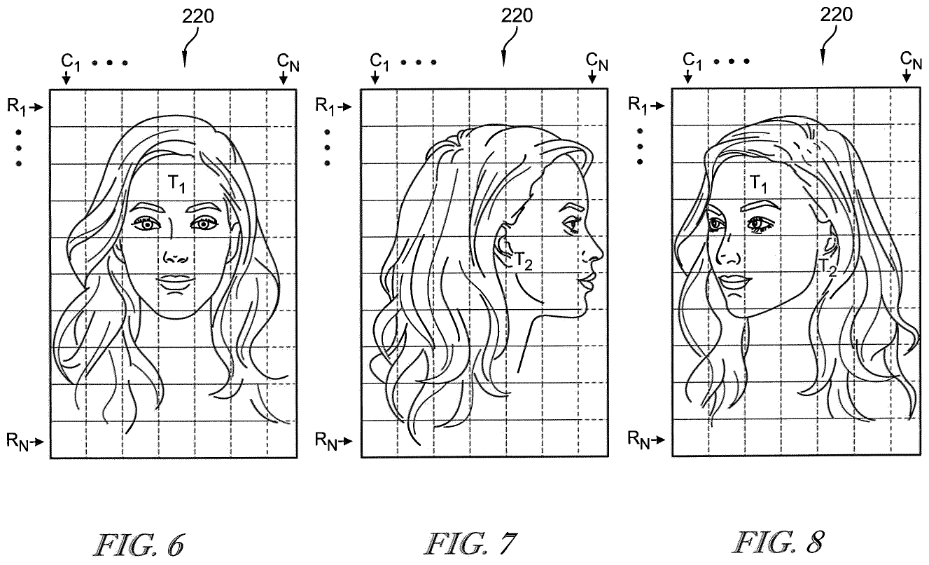

At process step 208, causes the targeting sensor 152 process the signal from the detector 160 to establish a field-of-view 220, shown in FIGS. 6-8, within which the head of a patient supported on the hospital bed 10 is expected to be found. The targeting sensor 152 using a time-of-flight approach to determine the distance of the target in the field-of-view from the detector 160, processes the information from the field-of-view 220 and segments the image into a number of columns C₁ to C_{N} and a number of rows R₁ to R_{N}. The detected distance is used to develop the number of columns and rows to be applied to the field-of-view. At step 210, and utilizing the algorithm taught through machine learning, the targeting sensor 150 is operable to distinguish features of the patient such that the area in which patient's forehead or earlobes are likely be positioned are identified. At decision step 212, the various locations are tested against an accuracy model to confirm that the location is a particular feature. If a given location is identified to be associated with a particular with sufficient accuracy based on an error detection scheme, the particular column and row associated with a feature is identified at process step 210 and the process 200 advances to process step 214. If a feature cannot be identified with sufficient accuracy, then the process 200 returns to process step 208 and a new image field is generated and processed with different columns and rows to further attempt to identify a feature.

At step 214, the temperature monitor 104 causes the infrared sensor 152 to acquire a peak temperature at the location identified with the feature at step 212. This temperature is processed by the temperature monitor in real-time and shared across the network 166. The process 200 then proceeds to step 216 where the temperature is logged by the diagnostic system 100 with a time stamp and the process 200 returns to the cycle start step 202.

It should be noted that the temperature monitor 104 is operable to process the image field 220 to determine an orientation of a patient such that different perspectives of the patient's head may be identified based on the nature of the boundaries of the patient's head as well as by determining the relationship of various features of the patient as compared to other features. In this way, a target for the infrared sensor 152 may be identified and the particular feature being targeted will be used by the temperature monitor 104 to convert the temperature reading taken by the infrared sensor 152 to an actual core temperature. For example, in the image of FIG. 6, the model may be readily able to identify the patient's forehead at T₁, but not the patient's earlobe at T₂. However, in FIG. 7, the model may not identify T₁ while being able to identify T₂. Still further, in FIG. 8, the orientation of the patient may allow for identification of both T₁ and T₂ such that the determination of the feature to be targeted by the infrared sensor 152 is based on which has the highest accuracy and best correlation to the actual core temperature of a patient.

In addition to the patient's core temperature, the diagnostic system 100 also acquires the patient's heart rate and respiratory rate through the cardiopulmonary sensor 102. The cardiopulmonary sensor 102 functions like the sensor for monitoring heart rate and respiratory rate disclosed in U.S. Pat. No. 11,172,892 titled “Patient Support Apparatus Having Vital Signs Monitoring and Alerting,” issued Nov. 16, 2021, which is incorporated herein for the disclosure of a contactless sensor for detecting heart rate and respiratory rate and the implementation on a patient support apparatus. The sensor 102 of the present disclosure is positioned below the mattress 52 and is operable to detect the heart rate and respiratory rate of a patient supported on the mattress 52 in real-time.

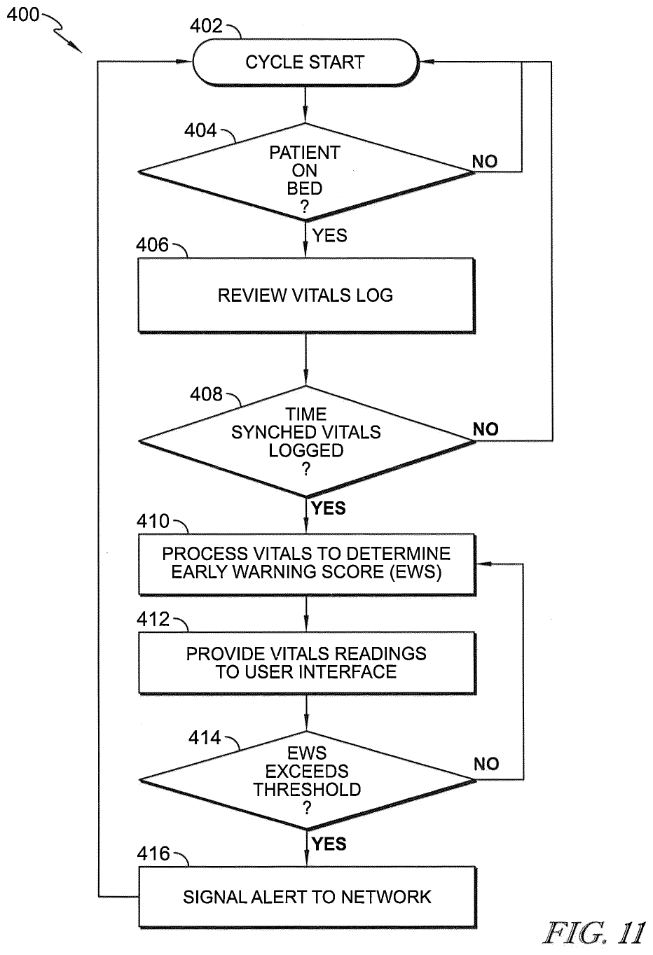

Referring to a process 300 shown in FIG. 10, the detection of cardiopulmonary vitals, e.g. heart rate and respiratory rate, starts with the cycle start at step 302. The process advances to a decision step 304 where information from the patient position monitor 114 is identified to determine whether or not a patient is present on the hospital bed 10. If the patient is determined not to be on the hospital bed 10, the process 300 returns to step 302 and repeats until patient is detected. If the patient is detected on the hospital bed 10 at step 304, the process advances to a step 306 where the signal is processed to determine the heart rate and respiratory rate of the patient. Those signals are provided to the network 166 in real-time. The process 300 then advances to decision step 308 where the signals are evaluated to determine if both the heart rate and respiratory rate are available. If they are not, then the process returns to the cycle start at step 302. If they are both available, the process 300 advances to step 310 where both the heart rate and respiratory are logged by the diagnostic system 100 with a time stamp.

The diagnostic system 100 continuously monitors the patient on the bed to evaluate the patient's EWS. As explained by Williams, “EWS [early warning score] systems assess the magnitude of physiological disturbance from the norm in response to acute illness and are generally based on allocating scores to routinely measured physiological parameters in a clinical setting. A score of zero representing no physiological disturbance and higher scores reflecting greater disturbance. By scoring multiple physiological parameters simultaneously and aggregating the score, the sensitivity and specificity of the score to detect acute deterioration earlier is enhanced.” Williams B. The National Early Warning Score: from concept to NHS implementation. Clin Med (Lond). 2022 November; 22 (6): 499-505. doi: 10.7861/clinmed.2022-news-concept. PMID: 36427887; PMCID: PMC9761416. (internal citations omitted).

According to the present disclosure, a process 400 for identifying and responding to the EWS is shown in FIG. 11. The process 400 begins at a cycle start at step 402. The process advances to a decision step 404 where information from the patient position monitor 114 is identified to determine whether or not a patient is present on the hospital bed 10. If the patient is determined not to be on the hospital bed 10, the process 400 returns to step 402 and repeats until patient is detected. If the patient is detected on the hospital bed 10 at step 404, the patient's vital signs log is reviewed at step 406. The process 400 then advances to decision step 408 where vitals log is evaluated to determine if time synched vitals have been logged and are available. If not, the process 400 continuously loops back to step 402. If time synched vitals have been logged and are available, then the process 400 advances to process step 410 where the vitals are processed to determine an EWS based on the logged vitals. This analysis includes reviewing the current vitals and the logged vitals as a time-series to evaluate the nature and magnitude of changes to determine the EWS. Once the EWS has been determined using a pre-defined approach, the current EWS is evaluated as compared to a predetermined threshold to determine if the EWS exceeds the predetermined threshold at decision step 414. If the EWS does not exceed the predetermined threshold at step 414, then the process 400 returns to step 410 and continues to determine the EWS in real-time.

If the EWS exceeds the predetermined threshold at decision step 414, the process 400 advances to process step 416 where the control system 118 receives an alert message that provides the EWS and identifies that an alert condition exists. This alert message may be used by the notification system 112 to provide an alert via the grip 76, the indicators 78, the indicators 82, and/or the user interface 62. The alert message is also communicated to the hospital network 136 such that the alert is shared with the caregiver mobile device 140 and the central monitoring system 138. At the central monitoring system 138, the alert may be displayed on a dashboard remote from the patient that provides a summary of the patient information, as is known in the art. Additionally, the alert message may be processed by the controller 120 to cause portions of the hospital bed 10 to change operation to mitigate the high EWS. This may include increasing the cooling effect of the mattress, moving portions of the bed to improve the heart rate or respiratory rate, initiating therapy functions, such as rotating the patient using continuous lateral rotation therapy and/or initiating percussion and vibration therapy.

The information from the diagnostic system 100 is made available to a user view the touchscreen display 110 on the FUD 66. The display 110 is segmented with an information banner 222 across the top which provides the status of bed systems and census information relative to the patient. A vitals display portion 226 is segmented to show a heart rate at 230, a respiratory rate at 240, and a temperature at 228. An acceptable upper heart rate limit for the particular patient is shown at 232 and the lower heart rate limit is shown at 234. Similarly the acceptable upper respiratory rate limit for the patient is shown at 242 and the lower respiratory rate limit is shown at 244. As shown in FIG. 12, the alarms for each of the vital signs are disabled. In some embodiments, if the EWS monitoring system is in use, the individual alarms may be disabled in favor of the unified EWS monitor.

The EWS is displayed at a banner 250. In the illustrative embodiment, the EWS is 5, which generates an alert condition which is indicated by the highlighting of banner 250 represented by the slashes 252. In some embodiments, this portion of the display 110 may be illuminated in red, may flash in red, or may operate in some other way to draw attention to a caregiver in proximity to the patient. Additionally, the notification system 112 may cause the grips 76, indicators 78, and indicators 82 to operate in an unusual manner, such as flashing red, or having all elements illuminated in red during an alert due to an excessive EWS.

In sum, the present disclosure provides an apparatus and method for monitoring patient vitals, including patient temperature, heart rate, and respiratory rate in real-time to evaluate the patient's EWS, thus providing early intervention. The intervention may include alerting caregivers as well as modifying operation of the hospital bed 10 to mitigate the factors leading to the EWS.

It is contemplated by the present disclosure that the processors 122, 132, 154, and 162 may be embodied as, or otherwise be included in, without limitation, an embedded computing system, a System-on-a-Chip (SoC), a multiprocessor system, a processor-based system, a microcontroller, an application specific integrated circuit (ASIC), a field programmable gate array (FPGA), and/or any other similar computing device. The present disclosure also contemplates that memory devices 124, 134, 156, and 164 may be embodied as one or more of volatile memory, non-volatile memory, random access memory (RAM), read only memory (ROM), a media disk, magnetic disk, optical storage, flash memory devices, and other similar devices capable of storing software for controlling functions. The memory devices 124, 134, 156, and 164 may be an independent device or integrated with any device serving as a processor 122, 132, 154, and 162.

It should be understood that the control system 118 may employ the RS-485 communications protocol over a twisted-pair bus. It should be understood that any of a number of communications protocols known in the art may be used for the communications between components of the control system 118, including, but not limited to, Echelon, CAN, SPI, USB, and LIN or another suitable electronic communications protocol. In still other embodiments, the communications may include circuitry that allows for a hardwired connection using an IEEE 802.3 connection, an RS-232 compliant connection, an RS-483 compliant connection, or other protocols known in the art including other wireless protocols. For example, the control system 118 may use of any one or more communication technologies (e.g., wired or wireless communications) and associated protocols (e.g., Ethernet, InfiniBand®, Wi-Fi®, WiMAX, 3G, 4G LTE, 5G, etc.) to effect communications between components within the control system 118 and to external systems.

Although this disclosure refers to specific embodiments, it will be understood by those skilled in the art that various changes in form and detail may be made without departing from the subject matter set forth in the accompanying claims. For example, while the disclosure has been illustrated and described in detail in the drawings and the foregoing description, such illustration and description are to be considered illustrative or exemplary and not restrictive. The disclosure is not limited to the disclosed embodiments. From reading the present disclosure, other modifications will be apparent to a person skilled in the art. Such modifications may involve other features, which are already known in the art and may be used instead of or in addition to features already described herein. In the claims, the word “comprising” does not exclude other elements or steps, and the indefinite article “a” or “an” does not exclude a plurality.

1. A patient support apparatus comprising
a diagnostic system including
a cardiopulmonary monitor detecting a patient's heart rate and respiratory rate without contacting the patient,
a temperature monitor comprising a targeting sensor and an infrared sensor, and a controller processing data from the targeting sensor to determine a location of a feature of a patient supported on the patient support apparatus, processing data from the infrared sensor in the location of the feature of the patient to determine the patient's body temperature, and
utilizing data from the cardiopulmonary monitor and the patient's body temperature to establish an early warning score for the patient, the diagnostic system comparing the early warning score for the patient to a threshold early warning score indicative of an imminent adverse event, and, if the early warning score exceeds the threshold, providing an output to initiate a therapeutic intervention to address the patient's condition.

2. The patient support apparatus of claim 1, wherein the therapeutic intervention to address the patient's condition includes altering a function of the patient support apparatus.

3. The patient support apparatus of claim 1, wherein the targeting sensor comprises a time of flight sensor.

4. The patient support apparatus of claim 1, wherein the feature of the patient is detected using a machine learning algorithm.

5. The patient support apparatus of claim 1, wherein the early warning score is displayed on a graphical user interface of the patient support apparatus.

6. The patient support apparatus of claim 1, wherein the early warning score is displayed on a central monitoring station that is positioned remotely from the patient's location.

7. The patient support apparatus of claim 1, wherein an output signaling to a caregiver to initiate a therapeutic intervention to address the patient's condition is displayed on a graphical user interface of the patient support apparatus.

8. The patient support apparatus of claim 1, wherein an output signaling to a caregiver to initiate a therapeutic intervention to address the patient's condition is transmitted to a central monitoring station.

9. The patient support apparatus of claim 1, wherein the temperature system is positioned on a movable portion of the patient support apparatus and the diagnostic system identifies the position of the movable portion of the patient support apparatus to determine if the temperature monitor is correctly positioned before attempting to determine a location of a feature of the patient.

10. The patient support apparatus of claim 1, wherein the diagnostic system comprises a processor, the processor operable to receive signals from the cardiopulmonary monitor indicative of the heart rate and the respiratory rate of the patient, signals from the infrared sensor indicative of the patient's temperature, and process the signals according to a predetermined relationship to determine the patient's early warning score in real time.

11. An early warning score monitoring system for a patient supported on a patient support apparatus comprising
a diagnostic system including a contactless cardiopulmonary monitor providing signals indicative of the heart rate and the respiratory rate of the patient, and a temperature monitor positioned on a movable portion of the patient support apparatus, the temperature monitor providing a signal indicative of the core temperature of the patient, the diagnostic system determining an early warning score of the patient in real-time, and
a notification system providing a notification to a caregiver if the real-time early warning score exceeds a predetermined threshold.

12. The early warning system of claim 11, wherein the temperature monitor includes a targeting sensor and an infrared sensor.

13. The early warning system of claim 12, wherein the targeting sensor comprises a time of flight sensor.

14. The early warning system of claim 11, wherein the targeting sensor identifies a feature of the patient and the temperature monitor identifies a temperature of the patient at the feature using the infrared sensor.

15. The early warning system of claim 14, wherein the temperature system is positioned on a movable portion of the patient support apparatus and the diagnostic system identifies the position of the movable portion of the patient support apparatus to determine if the temperature monitor is correctly positioned before attempting to determine a location of a feature of the patient.

16. The early warning system of claim 14, wherein the diagnostic system determines if the temperature monitor is correctly positioned before attempting to determine a location of a feature of the patient.

17. The early warning system of claim 11, wherein the contactless cardiopulmonary monitor is positioned on a frame member of the patient support apparatus.

18. The early warning system of claim 17, wherein the contactless cardiopulmonary monitor is positioned beneath a mattress supporting a patient on the patient support apparatus.

19. The early warning system of claim 11, wherein the contactless cardiopulmonary monitor is positioned beneath a mattress supporting a patient on the patient support apparatus.

20. The early warning system of claim 11, wherein, if the early warning score exceeds the threshold, the notification system provides an output to initiate a therapeutic intervention to address the patient's condition, wherein the therapeutic intervention to mitigate the patient's condition includes altering a function of the patient support apparatus.
